(19) United States
(12) Patent Application Publication
(10) Pub. No.: US 2026/0053592 A1
(43) Pub. Date: Feb. 26, 2026

(54) SYSTEMS, METHODS AND DEVICES FOR STEREOSCOPIC MICROFIBEROPTIC ROBOTIC SURGICAL VISUALIZATION

(71) Applicants: Eric Strand, Boston, MA (US); Noah Little, Boston, MA (US)

(72) Inventors: Eric Strand, Boston, MA (US); Noah Little, Boston, MA (US)

(21) Appl. No.: 19/271,424

(22) Filed: Jul. 16, 2025

Related U.S. Application Data
(60) Provisional application No. 63/685,157, filed on Aug. 20, 2024.

(51) Int. Cl. A61B 90/00; A61B 17/00; A61B 34/00; A61F 9/007; G06F 3/01; G06T 5/60; G06T 5/73; G06T 7/70

(52) U.S. Cl. CPC A61B 90/37; A61B 34/76; A61F 9/00736; G06T 5/60; G06T 5/73; G06T 7/70; A61B 2017/00398; A61B 2090/3614; A61B 2090/367; A61B 2090/373; G06F 3/012; G06F 3/013; G06T 2207/10012; G06T 2207/20192; G06T 2207/30004

(57) ABSTRACT

The present invention discloses a robotic surgical visualization system. The system comprises a robotic control unit, a wearable device, a user device, and a processing unit. The wearable device and the user device are configured to provide directional, rotational, and tactile control. The robotic unit is configured to capture a stereoscopic image by receiving an optical signal and converting the optical signals into digital imagery. The processing unit is in communication with the user device and the wearable device. The processing unit is further configured to receive the stereoscopic image from the control unit and transmit real-time stereoscopic image to the wearable device. Further, the wearable device comprises a visual-enhancement unit comprises an artificial intelligence (AI) module and a real-time stabilization module configured to enhance the stereoscopic image. The processing unit is further configured to store calibration data, and manages communication, image processing, calibration storage, and closed-loop feedback control.

### CROSS REFERENCE TO RELATED APPLICATION

The present disclosure claims priority to U.S. Provisional Application Ser. No. 63/685,157, filed Aug. 20, 2024, entitled “SYSTEMS, METHODS AND DEVICES FOR STEREOSCOPIC MICROFIBEROPTIC ROBOTIC SURGICAL VISUALIZATION”.

### BACKGROUND OF THE INVENTION

Surgical visualization is an essential component in modern minimally invasive procedures. The invasive procedures involve entering the body typically through a cut, puncture, small incisions or physiologic foramina to diagnose or treat a condition. The surgical visualization is utilized for critical visual feedback required for the safe and precise execution of operations. The invasive procedures, including vitreoretinal surgeries, require precise visualization particularly during surgery. The surgeries often require manipulation of delicate tissues at a sub-millimeter scale, where even slight deviations in visual clarity or stability could result in complications. Traditional surgical visualization systems typically rely on endoscopic or microscopic imaging systems. The traditional surgical visualization often provides a poor depth perception, low resolution, and reduced clarity of images of the surgical area in certain surgical fields.

Further, the existing surgical visualization includes a surgical microscope and monoscopic fiber endoscopes for invasive procedures. The existing surgical visualization offers valuable views of the surgical area but fails to provide the clear visualization required in modern microsurgical operations. In particular, the lack of clarity in the image could lead to limited depth perception. Further, the limited depth perception reduces the ability of surgeons to fully perceive the spatial relationships between delicate structures within the surgical area during surgery. The existing systems come with several limitations including low image resolution, instability during image capture, and challenges in fitting within the small incisions required for modern microsurgical techniques. Further, the traditional surgical microscopes and fiber endoscopes used in invasive procedures often fail to provide optimal depth perception, stability, and proper intraocular vantage points.

Therefore, there is a need for a robotic visualization system capable of capturing clear images that provide the depth perception and precision required for intricate surgical procedures. Accurate depth perception enables surgeons to fully understand the spatial relationships between delicate structures within the surgical field. Additionally, the system needs to provide a proper intraocular vantage point to support effective operation. Further, the system needs to be compact enough to fit within the small incisions required for modern microsurgical techniques.

### SUMMARY OF THE INVENTION

The present invention discloses a robotic surgical visualization system. The robotic surgical visualization system comprises at least one user device, at least one wearable device, a modular robotic control unit, and a processing unit in communication with the user device and the wearable device. The user device is associated with a user. The user device is further configured to provide the user input to the processing unit. The user input provides information related to directional movement control, rotational orientation control, general control input, and tactile feedback control to the robotic control unit. In one embodiment, the user device is a handheld controller. The user device is configured to control the movement and motion of the robotic control unit.

The modular robotic control unit comprises a base platform, at least one first base, a first housing, and a second housing. The base platform comprises one or more first apertures. The first aperture is configured to receive an external surgical system. The first base disposed on the base platform comprises a first sliding member and a second sliding member. The first sliding member and the second sliding member comprise at least one groove.

The first housing comprises at least one second base, a supporting member, one or more first motors, and a first pinion gear. The second base positioned on the first base comprises at least one first cavity and one or more appendages. The first cavity is configured to allow potential integration into a broader surgery platform. The first cavity comprises a first rack gear and a recessed channel configured to follow the curvature of the path and allow atraumatic movement of the robotic control unit about a center point of the atraumatic unit.

The appendages are configured on a bottom surface of the second base. The appendages are configured to secure within the first groove of the first sliding member and the second sliding member. The robotic control unit further comprises a first connector. The first connector is configured to the appendages and the groove of the first base. The first connector is configured to provide curvilinear movement between the appendages and the groove. The appendages could be a damper or spring-loaded mechanism. The appendages are configured to absorb shocks and vibrations induced during the movement. Further, the first connector is configured to reduce friction and provide stability during motor operation and repositioning of the motor.

The supporting member is disposed on the second base and comprises one or more second cavities at a rear portion of the supporting member. At least one second cavity is positioned at a lower portion of the supporting member, and at least one second cavity is positioned at an upper portion of the supporting member. The supporting member further comprises one or more second apertures. The second aperture is configured for one or more wires and connections required for the robotic control unit. The first motors positioned are within the second cavities of the supporting member. The first pinion gear is configured to position along the first sliding member. Further, the first pinion gear is engaged with the first motor and configured to transmit rotational motion. Further, the first pinion gear positioned with a first rack gear is configured to convert rotational motion from the motor into linear motion.

The second housing is disposed on the first housing of the robotic control unit. The second housing comprises a frame member, one or more fiber optic bundles, and a stereoscopic system. The frame member comprises one or more track channels, an anterior end, and a posterior end opposite to the anterior end. The anterior end is connected to a probe guide and the posterior end is configured to receive a guide system. The guide system comprises one or more third apertures, a probe tunnel, a circular track, and one or more pitch control motors. Each third aperture is configured to receive a threaded component. The threaded component is configured to secure the guide system to the frame member, thereby enabling the frame member to move along one dimension with minimal friction.

The probe tunnel is configured to receive an auxiliary system to support the visualization system. The auxiliary system includes but not limited to lighting, suction, or irrigation. Further, the probe tunnel is configured to end at the probe guide. The circular track is configured to receive a second pinion gear. The second pinion gear is engaged with at least one first motor configured to transmit rotational motion. The pitch control motor comprises a first pitch control motor and a second pitch control motor positioned adjacent to a flared opening at the posterior end of the frame member. The pitch control motors are configured to connect to one or more pitch control wires running along the robotic control unit.

The second housing further comprises a gear track. The gear track is configured to connect the pitch control motor and the first housing, thereby enabling pitch adjustment of the visualization system. Further, the gear track is configured to provide guidance to prevent misalignment of the pitch control motor. Further, the gear track is configured to ensure consistent and reliable performance over an extended period of the pitch control motor. The pitch control motor along the gear track enables bi-directional manipulation of the pitch control wires with high responsiveness.

The fiber optic bundles run along the supporting member and extend from the posterior end of the frame and pass through the anterior end of the frame member. The fiber optic bundle is configured to transmit an optical signal and an image data. The fiber optic bundles are configured to optimize light transmission, minimize optical aberrations, and deliver high-resolution imaging at sub-millimeter scales. The fiber optic bundles are configured to connect anteriorly to the probe guide and posteriorly to the stereoscopic system. The probe guide comprises a probe body and a probe cover.

The probe body comprises an opening configured to allow the fiber optic bundle to pass through. The probe body is configured to secure both the fiber optic bundle and the pitch control wire. The probe cover is configured to cover both the fiber optic bundle and the pitch control wire. Further, the probe cover is configured to support and direct the optical pathways created by the fiber optic bundles. The robotic control unit further comprises a disc member configured to surround the fiber optic bundles and interfaces with the pitch control wires. The disc member receives the force from the pitch control wire to bend the fiber optic bundles in the intended pitch direction. The disc member is configured to provide a stable and aligned base for the routing of the fiber optic bundles and the pitch control wires.

The stereoscopic system connected to the frame member comprises one or more optic imaging probes, a collimation assembly, and one or more cameras. The optic imaging probe coupled to the fiber optic bundle is configured to capture a real-time stereoscopic image of a surgical area. The collimation assembly is coupled to the fiber optic bundles, and the camera is configured to align the transmitted optical signals. The collimation assembly further comprises at least one collimating lens, at least one aspheric lens, at least one beam diffuser, one or more lens adjustment assemblies, and one or more beam expansion optics. Further, the lens adjustment assemblies are connected to the respective fiber optic bundles. The lens adjustment assemblies are configured to guide light from the surgical area of interest to the camera. The camera is further configured to convert transmitted optical signals into digital imagery. The camera is further configured to process the stereoscopic images using a computing unit to enhance visual clarity and depth perception.

The processing unit is disposed on the base platform and is in communication with the user device and the wearable device. The processing unit is configured to receive control signals and the user input from the user device and the wearable device. The processing unit is configured to provide output control signals to motors. The processing unit is further configured to receive the stereoscopic image from the robotic control unit and transmit the real-time stereoscopic image to the wearable device. The processing unit is configured to store calibration data, manage communication with the user device and the wearable device, and coordinate feedback loops for closed-loop control of the system.

The wearable device comprises a display unit, one or more sensors, and a visual-enhancement unit. The display unit is configured to display the captured stereoscopic images in real-time. The display unit is further configured to enable users to remotely control the robotic control unit to navigate the optic imaging probe via the wearable device. The display unit further comprises a communication interface with the visual-enhancement unit to dynamically adjust the display based on user movement. The sensor comprises a motion tracking sensor. Further, the sensor is configured to detect a head movement and an eye movement of the user. The wearable device is configured to provide hands free control functionality by translating the detected head movement and eye movement into a control signal. The control signals are configured to adjust the position and orientation of the optic imaging probe. The hands-free control functionality enables intuitive, real-time navigation within the surgical environment.

The visual-enhancement unit comprises an artificial intelligence (AI) module and a real-time stabilization module. The AI module is configured to analyze and optimize images. The AI module is further configured to generate a depth map from acquired stereoscopic image data in real-time. The AI module is further configured to filter noise from the image data. The AI module is further configured to perform image sharpening, contrast enhancement, and edge detection in real-time. The AI module is further configured to overlay anatomical highlights onto the processed image in real-time. The real-time stabilization module further comprises an inertial measurement unit (IMU) data and optical-flow fusion. The stabilization models are configured to minimize motion artifacts during image acquisition and process. The camera comprises a high-resolution camera coupled to the collimation assembly.

In one embodiment, a method of operation of the robotic surgical visualization system is disclosed. At one step, the processing unit is configured to guide the optic imaging probe through the robotic control unit based on the user input provided via the user device. At another step, the processing unit is further configured to actuate the robotic control unit using a rack-and-pinion mechanism to enable pivoting of the imaging probe about an atraumatic center point. The actuation of the robotic control unit is controlled manually via the user device, as well as hands-free through the wearable device. The sensors of the wearable device transmit the control signals to the system based on the head movement and eye movements of the user. Further, the signals from the sensor enable intuitive probe control and adaptive image presentation.

At yet another step, the processing unit is further configured to capture the stereoscopic image using the optic imaging probe positioned within a surgical site. At yet another step, the processing unit is further configured to transmit the stereoscopic images to the camera via the stereoscopic system. At yet another step, the processing unit is configured to receive the stereoscopic images from the camera. At yet another step, the wearable device is configured to enhance the captured images in real time using the artificial intelligence (AI) module. The artificial intelligence (AI) module is configured to enhance contrast, reduce visual noise, stabilize motion, and highlight anatomical features in real-time. At yet another step, the wearable device is further configured to process the stereoscopic image via the visual-enhancement unit to enhance visual clarity and depth perception. At yet another step, the wearable device is further configured to enable the display of the processed images to the user via the display unit.

### BRIEF DESCRIPTION OF THE DRAWINGS

FIG. 1 exemplarily illustrates a perspective view of a robotic surgical visualization system, according to an embodiment of the present invention.

FIG. 2 exemplarily illustrates a perspective view of a closed configuration of the robotic control unit, according to an embodiment of the present invention.

FIG. 3 exemplarily illustrates a perspective view of a robotic control unit, according to an embodiment of the present invention.

FIG. 4 exemplarily illustrates a top view of the robotic control unit, according to an embodiment of the present invention.

FIG. 5 exemplarily illustrates a first base, and a first motor disposed on a base platform of a robotic control unit, according to an embodiment of the present invention.

FIG. 6 exemplarily illustrates a second base, the first motor, the first base, and the base platform of the robotic control unit, according to an embodiment of the present invention.

FIG. 7 exemplarily illustrates a supporting member disposed on the base platform of the robotic control unit, according to an embodiment of the present invention.

FIG. 8 exemplarily illustrates a frame member disposed on the supporting member of the robotic control unit, according to an embodiment of the present invention.

FIG. 9 exemplarily illustrates a guide system received within the frame member disposed on the supporting member of the robotic control unit, according to an embodiment of the present invention.

FIG. 10 exemplarily illustrates one or more nuts fasten the frame member and the supporting member of the robotic control unit, according to an embodiment of the present invention.

FIG. 11 exemplarily illustrates one or more pitch control motors disposed on the posterior end of the frame member, according to an embodiment of the present invention.

FIG. 12 exemplarily illustrates a probe guide, a disc member, and a probe cover surrounding a fiber optic bundle and a pitch control wire, according to an embodiment of the present invention.

### DETAILED DESCRIPTION

The present invention is generally related to a surgical visualization system. More particularly, the invention relates to a system, methods, and devices for stereoscopic microfiber-optic robotic surgical visualization integrated with an artificial intelligence module to enhance the stereoscopic image. Non-limiting and non-exhaustive embodiments of the invention are described with reference to the following figures or drawings. Specifically, the embodiment below relates to the system and apparatus as they would be assembled for surgical visualization.

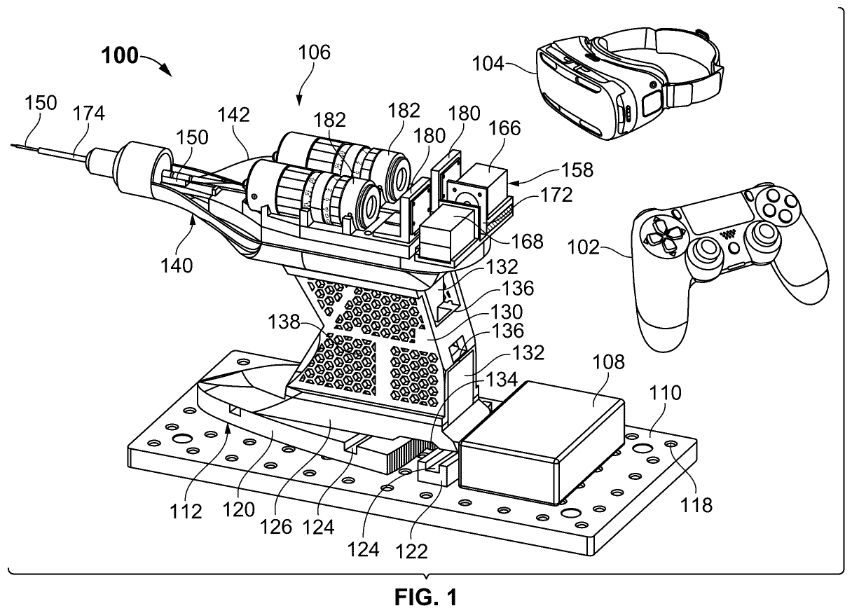

FIG. 1 exemplarily illustrates a perspective view of a robotic surgical visualization system 100, according to an embodiment of the present invention. The robotic surgical visualization system 100 comprises at least one user device 102, at least one wearable device 104, a modular robotic control unit 106, and a processing unit 108 in communication with the user device 102 and the wearable device 104.

The user device 102 is associated with a user. The user could be a surgeon. The user device 102 is configured to provide the user input to the processing unit 108. The user device 102 is configured to control the robotic control unit 106 manually. The user input provides information related to directional movement control, rotational orientation control, general control input, and tactile feedback control to the robotic control unit 106. In one embodiment, the user device 102 is a handheld controller. The handheld controller is a manual controller that allows the user to directly guide the movement of the robotic control unit 106 within a surgical area. The user device 102 is configured to control the movement and motion of the robotic control unit 106.

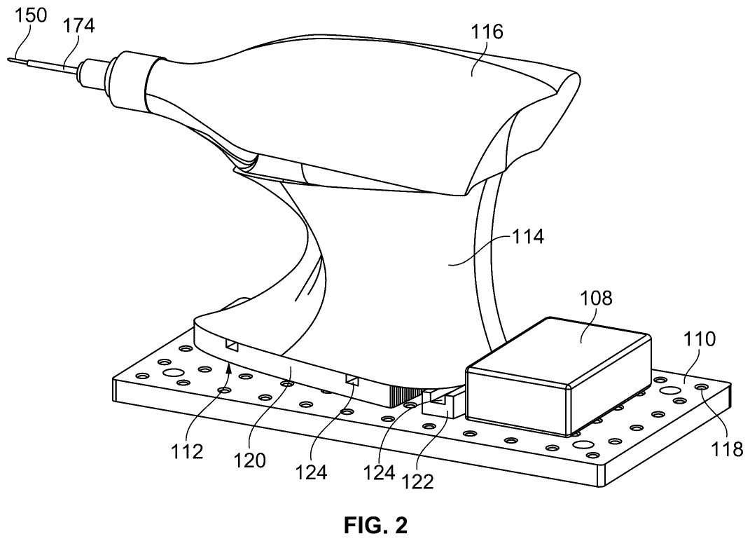

Referring to FIG. 2, the modular robotic control unit 106 comprises a base platform 110, at least one first base 112, a first housing 114, and a second housing 116. The first housing 114 and the second housing 116 are configured to cover and shield the internal components of the robotic control unit 106. The first housing 114 and the second housing 116 are configured to provide structural integrity to the robotic control unit 106 and help the robotic control unit 106 withstand mechanical stress and impacts. The first housing 114 and the second housing 116 are further configured and designed to facilitate easy assembly and maintenance. The first housing 114 and the second housing 116 are further configured to provide ease access to internal components when necessary. The second housing 116 is positioned on the top portion of the robotic control unit 106. The second housing 116 is designed to shield internal components while also contributing to the ergonomic form of the apparatus and ensures the second housing 116 fits comfortably and securely during use. The first housing 114 of the robotic control unit 106 complements the second housing 116 by enclosing the underside, thereby completing the protective housing.

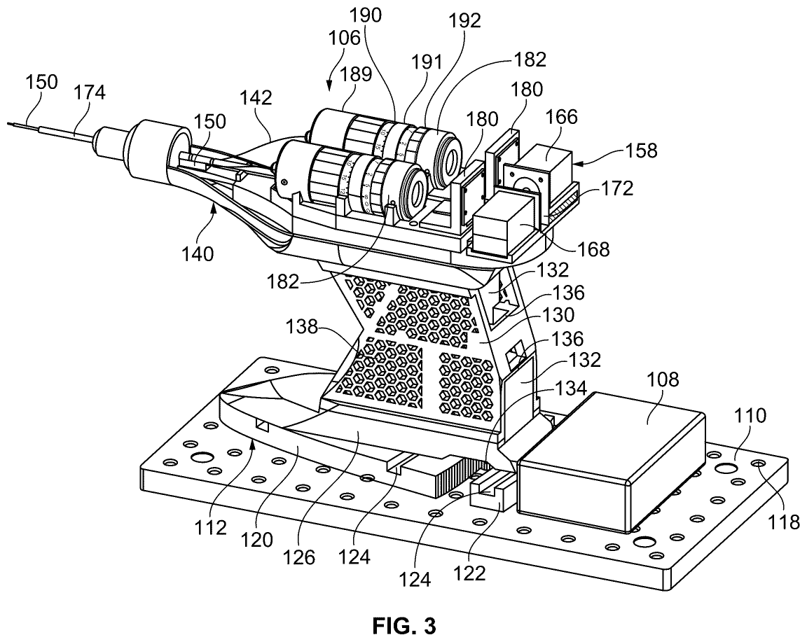

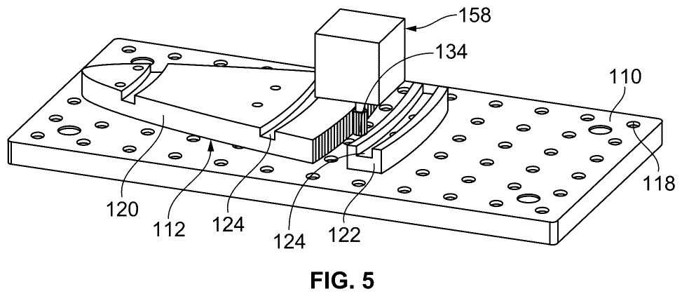

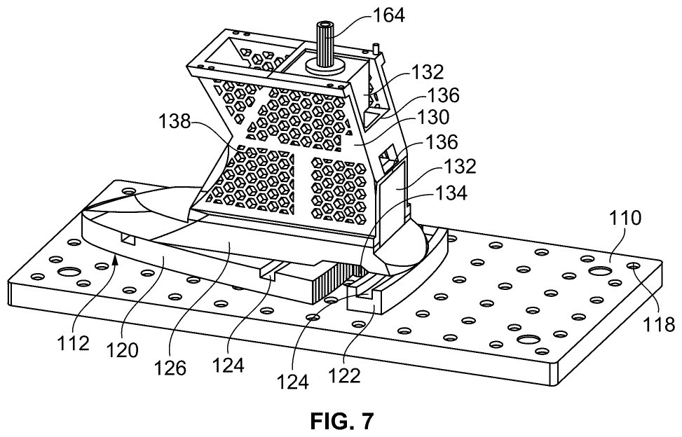

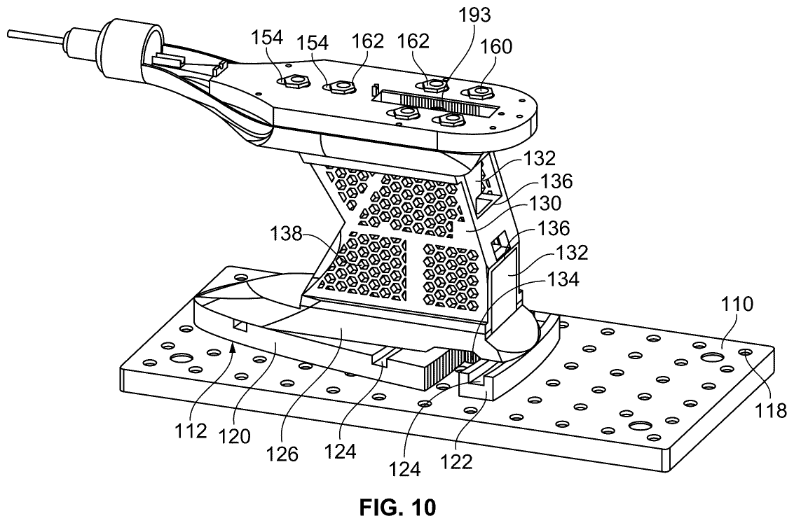

Referring to FIG. 1, FIG. 2, FIG. 3, FIG. 5 to FIG. 11, the base platform 110 comprises one or more first apertures 118. The first aperture 118 is configured to receive an external surgical system. The first base 112 is disposed on the base platform 110 and comprises a first sliding member 120 and a second sliding member 122. The first sliding member 120 and the second sliding member 122 comprise at least one groove 124.

The first housing 114 comprises at least one second base 126, a supporting member 130, one or more first motors 132, and a first pinion gear 134. Referring to FIG. 1, FIG. 3, FIG. 5 to FIG. 11, the second base 126 is positioned on the first base 112 and comprises at least one first cavity 128 and one or more appendages. The first cavity 128 is configured to allow potential integration into a broader surgery platform. The first cavity 128 comprises a fist rack gear and a recessed channel configured to follow the curvature of the path and allow atraumatic movement of the robotic control unit 106 about the center point of an atraumatic unit 186.

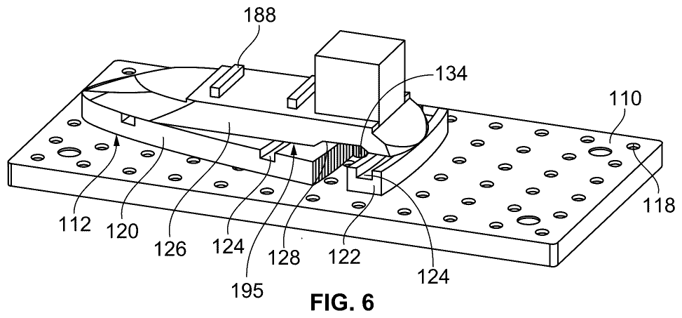

The appendages 188 are configured on a bottom surface of the second base 126. The appendages could be a damper or spring-loaded mechanisms. The appendages 188 are configured to absorb shocks and vibrations induced during the movement. The appendages are configured to secure within the first groove 124 of the first sliding member 120 and the second sliding member 122. The appendages are configured to maintain stability while allowing guided motion. The robotic control unit 106 further comprises a first connector 195. The first connector 195 is configured to the appendages 188 and the groove 124 of the first base 112. The first connector 195 is configured to provide curvilinear movement between the appendages and the groove 124 (see FIG. 6). Further, the first connector is configured to reduce friction and provide stability during motor operation and repositioning of the motor. 195 reference underside connector appendages, acting as pegs that stick down into the center of a ball bearing (laying on its side) to provide smooth curvilinear movement in grooves 124. There are two appendages/ball bearing sets per groove.

The first connector could be a ball bearing or other friction reducing connectors could be used resulting in movement along the circular path 184. The smooth linear or curvilinear movement along the circular path 184 is facilitated by one or more ball bearings, roller elements or other low-friction connectors within the channel surfaces and the appendages of the second base 126. The first sliding member 120 and the second sliding member 122 limits the movement of the apparatus to circular path 184 which ensures the tip of the atraumatic unit 186 of the robotic control unit 106 remains focused on the target site of the surgical area. The atraumatic unit 186, the circular path 184, and the processing unit 108 are structural and functional elements of the system 100, configured to coordinate spatial orientation, motion tracking, and data processing functionalities.

The center point of the atraumatic unit 186 is fixed and dynamically calculates the reference position within the system 100, from which angular orientation, displacement, or movement of the robotic control unit 106 components might be measured or guided. The center point of the atraumatic unit 186 serves as a spatial anchor to facilitate precise control and repeatable motion, particularly when rotational or accurate movement is required. In one embodiment, the center point of the atraumatic unit 186 might correspond to a pivot or fulcrum about which certain device components rotate or articulate.

The circular pathing is defined relative to the center point and represents the circular path 184 along which a movable component or visualization element is configured to travel. The circular path 184 might be mechanically constrained through structural guides, tracks, or linkages, or might be defined algorithmically via the robotic control unit 106. The mechanical containment is configured to regulate articulation based on the user input or pre-programmed motion profiles. The circular path 184 is configured to enable consistent and predictable movement around the center point of the atraumatic unit 186. The circular path 184 is configured to support and enhance navigational accuracy and positional feedback within the system 100.

The supporting member 130 is disposed on the second base 126 and comprises one or more second cavities 136 at a rear portion of the supporting member 130. At least one second cavity 136 is positioned at a lower portion of the supporting member 130, and at least one second cavity 136 is positioned at an upper portion of the supporting member 130. The supporting member 130 is designed as a honeycomb structure for material savings and strength. The supporting member 130 reduces the quantity of necessary materials for the component as well as reduces the overall weight of the system 100. The supporting member 130 is configured to provide high strength and rigidity with minimal weight. Further, the supporting member 130 securely houses and provides stabilizing support for the first motor 132. The supporting member 130 acts as a mounting platform, allowing the first motor 132 to exert force on the connected first pinion gear 134.

The supporting member 130 further comprises one or more second apertures 138. The second aperture 138 is configured for one or more wires and connections required for the robotic control unit 106. The second aperture 138 is the strongest shape due to the ability to evenly distribute loads of the robotic control unit 106 across the perimeter of the shape, by emulating the shape of a circle through the honeycomb structure. The forces on the supporting member 130 are more evenly distributed across the whole perimeter compared to a square. The supporting member 130 also offers a higher area to perimeter ratio compared to a square, meaning less materials are necessary when using this honeycomb structure.

Wired connections from motors 132, 166, and 168 pass through openings in frame 130 to attach to processing unit 106. The set of wired connections for each motor includes a power wire, ground wire, afferent control data wire, and efferent positional data wire. The exact gauge, voltage, and amperage carried by the wires may vary with motor manufacturer and may be provided dynamically by an appropriate and commercially available power supply located within the processing unit.

Wired connections from cameras 180 pass through openings in frame 130 to attach to processing unit 106. The set of wired connections for each camera include a power connection (with integrated power and ground), and an efferent imaging data connection (for example, USB or HDMI protocol). Power is supplied by the manufacturer specific power supply for each camera or may be provided by an appropriate and commercially available power supply located within the processing unit.

Connections from the processing unit to the wearable headset may take both wired and wireless forms. In a wired embodiment, the processed stereoscopic imaging information and any overlay/HUD information is carried by a high-speed imaging connection (for example microHDMI) from the processing unit to the headset. Head-tracking information may be conveyed by a separate connection (for example USB) to provide real-time control information from the headset to the processing unit. In a wireless embodiment, the processing unit may instantiate a low-latency Bluetooth or WiFi signal that enables bi-directional information transfer to and from the headset. Through this connection, processed imaging information is transmitted to the headset and real-time control information is transmitted back from the headset. In embodiments where the user desires to use the handheld control device, wired (typically USB) or wireless (typically Bluetooth) connections may be used to convey control information from the handheld device to the processing unit.

The first motor 132 is positioned within the second cavities 136 of the supporting member 130. The first pinion gear 134 is configured to position along the first sliding member 120. Further, the first pinion gear 134 is engaged with the first motor 132 and is configured to transmit rotational motion. Further, the first pinion gear 134 is positioned with a first rack gear and configured to convert rotational motion from the motor into linear motion.

The supporting member 130 is configured to provide support to the first motor 132 which drives the first pinion gear 134. The first motor 132 and the first pinion gear 134 interface with the first rack gear of the first sliding member 120. As the first motor 132 turns the first pinion gear 134, the gear stays within the first rack gear ensuring movement along the first sliding member 120 and the second sliding member 122. Both the first rack gear and the recessed channels are designed to follow the circular path 184 that allows the structural components to be attached to the recessed channels and enable atraumatic movement of the system 100. The first motor 132 and the first pinion gear 134 with the first rack gear are part of the ability of the system 100 to move atraumatic movement at the center point of the atraumatic unit 186.

The first motor 132 is connected to the first pinion gear 134 or “rack-and-pinion mechanism” which serves the primary function of generating rotational motion and mechanical force, which is exerted upon and drives the first pinion gear 134. The first pinion gear 134 is configured to serve as a central mechanism for controlling the positioning of the system 100 along the first sliding member 120 and the second sliding member 122. The robotic control unit 106 is configured to utilize the force provided by the first motor 132. The first pinion gear 134 rotates in response to the user input and configured to adjust the position of the robotic control unit 106 accordingly. The movement is achieved by turning the first pinion gear 134 along the grooves 124 located in the first sliding member 120 and the second sliding member 122. The relationship between the user inputs and the rotation output of the motor, which drives the pinion gear is a feature of the system 100. The robotic control unit 106 is configured to allow the subject to be viewed from different angles based on the user inputs.

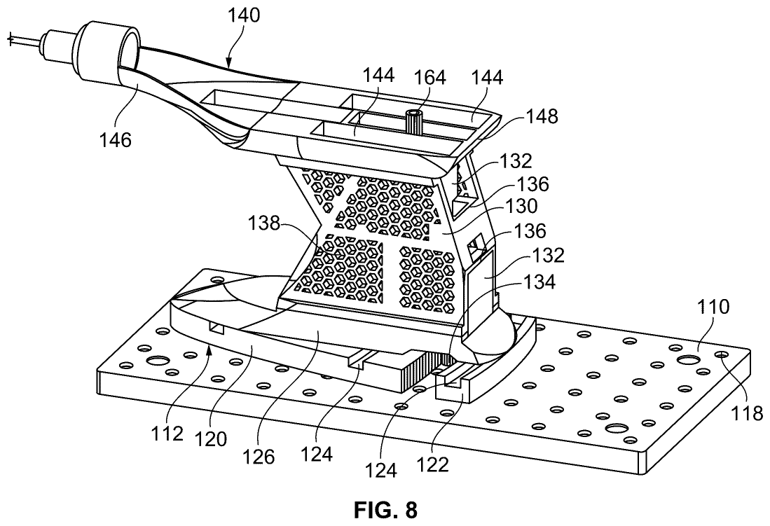

Referring to FIG. 1, FIG. 3, FIG. 8 to FIG. 11, the second housing 116 is disposed on the first housing 114 of the robotic control unit 106. The second housing 116 comprises a frame member 140, one or more fiber optic bundles 142, and a stereoscopic system. The frame member 140 comprises one or more track channels 144, an anterior end 146, and a posterior end 148 opposite to the anterior end 146. The anterior end 146 is connected to a probe guide 150 and the posterior end 148 is configured to receive a guide system 152. The probe guide 150 is a static structure designed to cover components which are directed at the target site of the surgical area. The probe guide 150 allows the fiber optic bundles 142 to reach the site of interest within the surgical area.

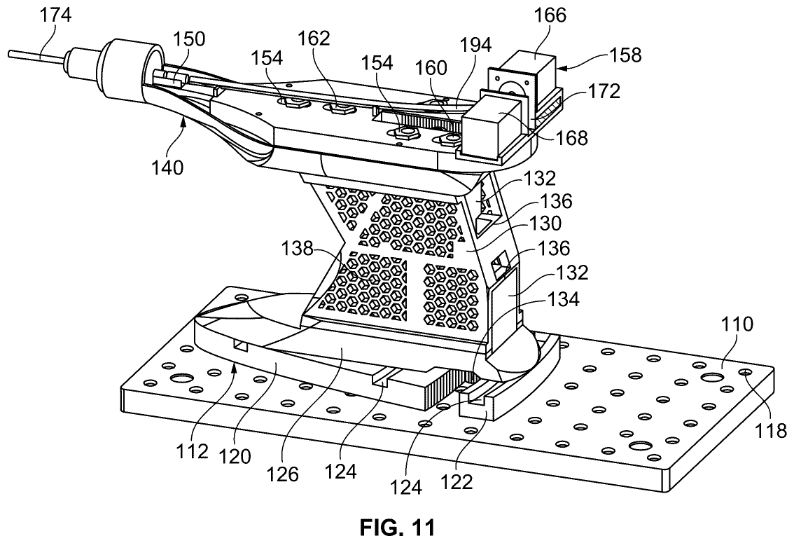

The anterior end 146 of the frame member 140 comprises an elongated guide (see also FIG. 11, 174) that extends to the atraumatic unit 186. The elongated guide is hollow and allows the optic fiber bundle 142 to travel through a central opening through which the pinion gears could traverse. The track channels 144 along the supporting member 130 are configured to allow minimally the guide system 152 to remain stable and undisturbed. The frame member 140 is configured to guide the guide system 152 by directing the optical pathway necessary for imaging the surgical area. Integrated within the frame member 140 are recesses, which house the frictionless track channel 144 designed to allow the guide system 152 to slide forward and backward, thereby enabling controlled advancement of the camera 180, lens, or probe apparatus along a single axis.

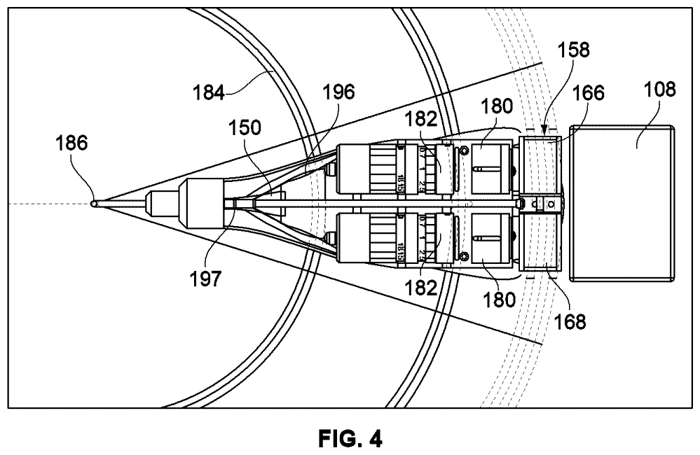

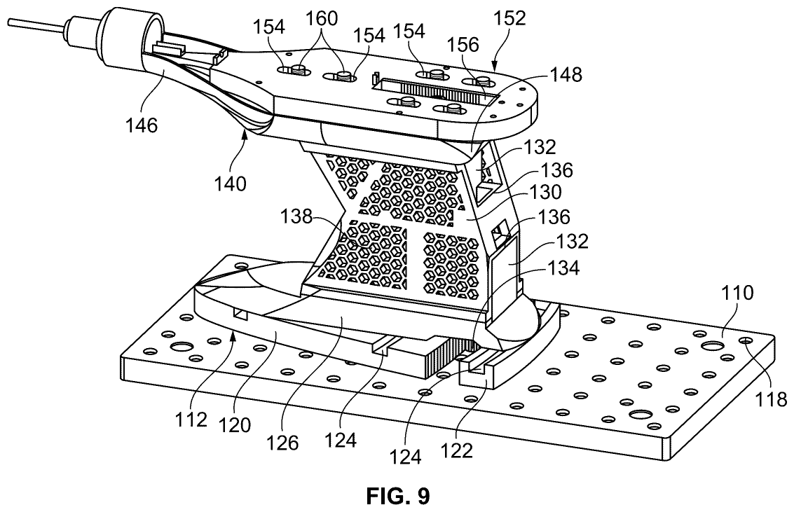

Referring to FIG. 1, FIG. 3, FIG. 4, FIG. 9 to FIG. 11, the guide system 152 comprises one or more third apertures 154, a probe tunnel 156, a circular track, and one or more pitch control motors 158. Each third aperture 154 is configured to receive a threaded component 160. The threaded component 160 is configured to secure the guide system 152 to the frame member 140, thereby enabling the frame member 140 to move along one dimension with minimal friction. The guide system 152 having the third aperture 154 interfaces with the raised threaded sections of the minimally frictional slide base below. The guide system 152 is referred to as a sliding base. The threaded components 160 is configured to secure the guide system 152 to the frame member 140 when tightened using one or more nuts 162. The circular track is configured to receive a second pinion gear 164. The design of threading and threaded components 160 allows the frame member 140 to move smoothly in one direction through the action of the probe tunnel 156 and the second pinion gear 164.

The probe tunnel 156 interfaces with the second pinion gear 164. The guide system 152 also contains support sections for visualization system 100 on the anterior end 146 of the frame member 140. Lubricating agents are added to the guide system 152 to reduce friction. The lubricating agents include, but are not limited to, grease, oil, or silicone. The lubricated agents allow for seamless transition of the guide system 152 back and forth. The lubricated agent is configured to enhance a linear direction, increased stability and responsiveness, and reduces wear on the robotic control unit 106.

The probe tunnel 156 is configured to receive an auxiliary system 194 to support the robotic control unit 106. 194 references end of probe guide where additional auxiliary systems 194 would interface; the end is obscured by motors 166 and 168 in FIG. 11. The auxiliary system 194 includes, but is not limited to, lighting, suction, or irrigation. Further, the probe tunnel 156 is configured to end at the probe guide 150. The circular track that receives the second pinion gear 164 is configured to engage with at least one first motor 132, configured to transmit rotational motion. The probe tunnel 156 comprises a second rack gear 193. The probe tunnel 156 interfaces with the second pinion gear 164 and allows for control of the movement of the guide system 152 as the second pinion gear 164 interfaces with the probe tunnel 156 of the central recess. In the rack and pinion system, the rotary motion of the second pinion gear 164 is converted into the linear motion of the second rack gear 193. Further, the rack and pinion system work with the guide system 152 located on the track channels 144 of the frame member 140. The rack and pinion system is configured to allow control of the linear motion of the frame member 140. The mechanism enables precise linear positioning of the guide system 152 in a linear direction, which directly affects the positioning of the visualization system 100.

The pinion track system operates in concert with the modular guide rails to restrict motion to a single translational axis, minimizing undesired play and ensuring precision control. The alignment is critical for procedures requiring consistent directional movement of surgical tools or visualization apparatus. The interface between the threaded components 160 and guide rails ensures a secure yet smooth path for mechanical elements during operation, thus enhancing stability and control in minimally invasive environments.

The system 100 relies on the precision alignment of mechanical interfaces to avoid undesired friction or slippage during the guidance of the probe. By tightly binding the frame member 140 to the minimally guide system 152 using the threaded components 160, the system 100 achieves a mechanically stable yet dynamically responsive configuration. The system 100 is critical when integrating with the probe tunnel 156. The system 100 further ensures the gear rotation is directly translated into controlled linear motion without backlash. The configuration allows the system 100 to accommodate a range of modular attachments and additional mechanical structures without compromising its core motion mechanics. The use of standardized threading and geometric symmetry enhances manufacturability and facilitates integration with other surgical or imaging platforms. The system 100 supports both manual and remote operation, allowing it to adapt to different procedural contexts or surgical preferences.

The pitch control motor 158 comprises a first pitch control motor 166 and a second pitch control motor 168 positioned adjacent to a flared opening at the posterior end 148 of the frame member 140. The first pitch control motor 166 is a positive motor and the second pitch control motor 168 is a negative motor. The positioning of the first pitch control motor 166 and the second pitch control motor 168 are configured to enable bi-directional manipulation of the pitch control guide wires 170 with high responsiveness. The close proximity of the pitch control motors 158 to the flared opening ensures minimal latency and slack in the movement of components passed through the probe tunnel 156. Further, the probe guide 150 serves both protective and alignment roles, keeping key control elements shielded while directing them efficiently toward the site of interest, allowing seamless integration with adjacent subsystems. The pitch control motors 158 are configured to connect to one or more pitch control wires 170 running along the robotic control unit 106. The pitch control wires 170 are mechanically connected to one or more movable components within the robotic control unit 106 and are configured to facilitate angular articulation or directional adjustment.

Each pitch control motor 158 is configured to engage directly with the respective guide wire, ensuring independent actuation along the positive and negative axes. The bilateral control allows the optic imaging probe 176 or associated instruments to pivot with refined angular resolution, offering improved dexterity within confined anatomical spaces. The optic imaging probe 176 continues back inside the probe body 174 and attaches to the frame member as seen in FIG. 3, 150.

The track channel 144 is configured to provide structural guidance to prevent drift or misalignment of the pitch control motor 158. The track channel 144 is further configured to enable repeatable and reliable performance during prolonged use. The track channel 144 is designed to streamline the frame member 140 by reducing the need for external cabling or secondary lumens. The integration of the probe tunnel 156 within the guide system 152 underscores an innovative design focused on compactness, ergonomic integration, and surgical efficiency.

The end of the probe guide 150 comprises a variety of gauges. The system 100 is configured to utilize the 20-gauge (G) standard, which has an outside diameter of 0.908 millimeters. The preferred gauge standards allow for two fiber optic bundles 142, each containing approximately 10,000 fibers, to be accommodated within the probe guide 150. Smaller gauge sizes include 23G, 25G, and 27G with outer diameters of 0.642 millimeters, 0.515 millimeters, and 0.413 millimeters respectively. The number of fiber optics in each bundle may be reduced to fit within the more constrained space when using these smaller diameters.

The second housing 116 further comprises a gear track 172. The gear track 172 is configured to connect the pitch control motor 158 and the first housing 114, thereby enabling pitch adjustment of the system 100. Further, the gear track 172 is configured to provide guidance to prevent misalignment of the pitch control motor 158. Further, the gear track 172 is configured to ensure consistent and reliable performance over an extended period of the pitch control motor 158. The pitch control motor 158 along the gear track 172 enables bi-directional manipulation of the pitch control wires 170 with high responsiveness.

The fiber optic bundle 142 runs along the supporting member 130 extend from the posterior end 148 of the frame member 140 and passes through the anterior end 146 of the frame member 140. The fiber optic bundle 142 is configured to transmit an optical signal and an image data. The fiber optic bundle 142 is configured in a left and right imaging channel for three-dimensional capture. The fiber optic bundles 142 are configured to optimize light transmission, minimize optical aberrations and deliver high-resolution imaging at sub-millimeter scales. The fiber optic bundle 142 comprises one or more optical fibers capable of providing illumination to convey visual information from the target site to the wearable device 104 located elsewhere in the system 100.

The fiber optic bundle 142 is a multimodal fiber optic bundle. In one embodiment, the system 100 utilizes a dual fiber bundle. Each fiber bundle contains approximately 10,000 pixels and has a diameter of 0.35 millimeters (mm). In one embodiment, the fiber bundles are supplied by Sumita Optical Glass Inc. (Saitama, Japan) and are each comprised of 10,000 individual fibers of 0.327 mm diameter surrounded by approximately 0.03 mm of jacket (product number 7L7A001). Each fiber bundle may be married to a distal 120-degree field of view lens of approximately 0.37 mm diameter and 3 mm length to assist in focusing incoming light.

The fiber bundle 142 is configured to enable detailed visualization at microscale levels. The fiber optic bundles 142 may comprise the lens of the same diameter as the bundles at the furthest anterior aspect to help guide light into the fiber optic bundle 142 from the area of interest. The system 100 employs dual image channels to produce the depth information necessary for microsurgical procedures. By overlaying the left and right image streams, the system 100 generates a unified stereoscopic image of the surgical cavity, providing the depth perception essential for precise microsurgery.

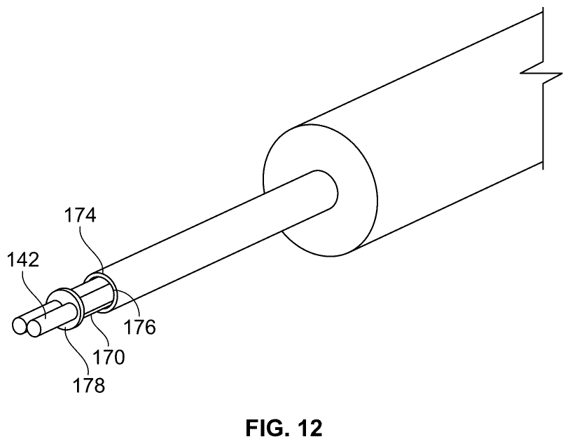

The fiber optic bundles 142 are configured to connect anteriorly to the probe guide 150 and posteriorly to the frame member 140. Referring to FIG. 12, the probe guide 150 comprises a probe body 174 and a probe cover 176. The probe body 174 comprises an opening configured to allow the fiber optic bundle 142 to pass through. The probe body 174 is configured to secure both the fiber optic bundle 142 and the pitch control wire 170. The probe cover 176 is configured to cover both the fiber optic bundle 142 and the pitch control wire 170. Further, the probe cover 176 is configured to support and direct the optical pathways created by the fiber optic bundles 142. In one embodiment, the probe cover 176 comprises one or more structural supports designed to maintain the alignment and orientation of the optic fiber bundle 142, thereby preserving image quality and minimizing signal distortion during use.

The robotic control unit 106 further comprises a disc member 178. The disc member 178 is configured to surround the fiber optic bundles 142 and interfaces with the pitch control wires 170. The disc member 178 receives the force from the pitch control wire 170 to bend the fiber optic bundles 142 in the intended pitch direction. The disc member 178 is operatively coupled to the fiber optic bundle 142 and the pitch control wire 170 and may function as a mounting platform or rotational interface for associated components. In one embodiment, the disc member 178 is configured to provide a stable and aligned base for the routing of the fiber optic bundles 142 and the pitch control wires 170. The disc member 178 is configured to allow the entire system 100 to track through the supporting structure, and further about the atraumatic unit 186. The disc member 178 is configured to serve to constrain or guide the movement during operation. The pitch control wires 170 might be routed through the disc member 178 and extend distally toward an articulating segment, where tensioning of the pitch control wires 170 enables controlled deflection or steering. Collectively, the components enable precise navigation and real-time visualization capabilities within the robotic control unit 106, particularly in applications requiring controlled access to confined or anatomically complex environments.

The frame member 140 further comprises one or more optic imaging probes, a collimation assembly, and one or more cameras 180. The optic imaging probe 176 is referred to as a microfiber probe. The optic imaging probe 176 is coupled to the fiber optic bundle 142 and configured to capture a real-time stereoscopic image of the surgical area (see FIGS. 3 and 12). The optical imaging probe is configured to be controlled manually via the user device 102 and as well as hands-free through the wearable device 104. In one embodiment, the imaging probe comprises an outside diameter of approximately 0.91 millimeters at a 20-gauge scale. In one embodiment, the imaging probe comprises roughly 20,000 individual fibers supplied by Sumita Optical Glass Inc. (Saitama, Japan) which enables capture of high-resolution images even in confined surgical areas.

The collimation assembly coupled to the fiber optic bundles 142, and the camera 180 is configured to align the transmitted optical signals. The collimation assembly 189 further comprises at least one collimating lens 190, at least one aspheric lens 191, at least one beam diffuser 192, one or more lens adjustment assemblies 182, and one or more beam expansion optics (see FIG. 3). The beam expansion components allow for the best output images from the lens adjustment assembly 182 and translate to the best image quality of the system 100.

Further, the lens adjustment assemblies 182 are connected to the respective fiber optic bundles 142. The lens adjustment assemblies 182 are configured to guide light from the surgical area of interest to the camera 180. The lens adjustment assemblies 182 coupled to the fiber optic 142 bundle are designed for high-fidelity image transmission, which is crucial for real-time visualization in diagnostic or surgical applications. Each component within the lens is selected to contribute to overall image clarity and focus versatility. Thereby enhancing depth perception in the stereoscopic output.

The camera 180 is further configured to convert transmitted optical signals into digital imagery. The camera 180 is further configured to process the stereoscopic images using a computing unit to enhance visual clarity and depth perception. The camera 180 is utilized for both the left and right track channel 144 of the robotic control unit 106. The dual-camera configuration ensures that both left, and right visual channels maintain optimal parallax and focus, which is essential for accurate 3D visualization during fine manipulations. Further, the camera 180 is an interface with assembly in an adjustable fashion to allow for optimal alignment of the camera 180 with the output of lens adjustment assemblies 182. The modular configuration of the camera 180 and lens adjustment assembly 182 allows for on-the-fly adjustments or replacements without dismantling the entire robotic control unit 106. This is especially valuable in intraoperative settings, where maintaining system 100 uptime is critical. Additionally, the camera 180 is mounted using adjustable fixtures to ensure precise optical alignment with the focal output of the lens systems, maximizing the fidelity and depth of the stereoscopic imagery.

The processing unit 108 is disposed on the base platform 110 is in communication with the user device 102 and the wearable device 104. The processing unit 108 is configured to receive control signals and the user input from the user device 102 and the wearable device 104. The processing unit 108 is configured to provide output control signals to motors. The processing unit 108 is further configured to receive the stereoscopic image from the robotic control unit 106 and transmit the real-time stereoscopic image to the wearable device 104.

The processing unit 108 is an electronic component operatively coupled to the various sensors, actuators, and data transmission elements of the system 100. The processing unit 108 is configured to receive, process, and interpret input signals, including visual data from the fiber optic bundles 142, positional data from movement tracking components, and user input from user device 102. The processing unit 108 is configured to execute algorithms for image enhancement, articulation control, real-time visualization rendering, or other computational tasks required for system functionality. Additionally, the processing unit 108 is configured to store calibration data, manage communication with the user device 102 and the wearable device 104, and coordinate feedback loops for closed-loop control of the system 100.

The processing unit 108 is connected to the wearable device 104 via a wired connection. In one embodiment, the processing unit 108 is connected to the wearable device 104 via a wireless connection. The wearable device 104 receives processed images from the robotic control unit 106 via the processing unit 108. The wired connection comprises a Universal Serial Bus (USB) 3.2 Generation 1, which offers a theoretical transfer speed of 5 gigabits per second (Gb/s) and a practical transfer speed of approximately 3.2 gigabits per second. Alternatively, the wearable device 104 might be equipped with wireless communication capabilities includes, a Quest 3 headset, which supports Wireless Fidelity 6 Extended (Wi-Fi 6E). Wi-Fi 6E could provide data transfer rates of up to 9.6 gigabits per second, with typical operational rates of around 1 gigabit per second. The latency for both wired and wireless connections may be as low as 2 milliseconds (ms). Further, the system 100 ensures effective use during surgical procedures by maintaining image transmission latency below 10 milliseconds.

The wearable device 104 comprises a display unit, one or more sensors, and a visual-enhancement unit. The display unit is configured to display the captured stereoscopic images in real-time. The display unit is further configured to enable users to remotely control the robotic control unit 106 to navigate the optic imaging probe via the wearable device 104. The display unit further comprises a communication interface with the visual-enhancement unit to dynamically adjust the display based on user movement. The sensor comprises a motion tracking sensor. Further the sensor is configured to detect a head movement and an eye movement of the user. The wearable device 104 is configured to provide hands free control functionality by translating the detected head movement and eye movement into one or more control signals. The control signals are configured to adjust the position and orientation of the optic imaging probe. The hands-free control functionality enables intuitive, real-time navigation within the surgical environment. The display unit provides the user with an immersive view of the surgical field. The wearable device 104 having the motion tracking capabilities allows the system to precisely track the head movement and the eye movements of the user.

The visual-enhancement unit comprises an artificial intelligence (AI) module and a real-time stabilization module. The AI module is configured to analyze and optimize images. The AI module is further configured to generate a depth map from acquired stereoscopic image data in the real time. The real-time stereo depth mapping techniques include Semi-Global Matching or might implement neural networks including “LightEndoStereo” or “StereoMamba” to enhance results. The stereo depth mapping technique is configured to produce a disparity map for each stereo frame with a latency of less than 20 milliseconds. The AI module is further configured to filter noise from the image data. The AI module is further configured to perform image sharpening, contrast enhancement, and edge detection in real time. The AI module is further configured to overlay anatomical highlights onto the processed image in real time. The real-time stabilization module further comprises an inertial measurement unit (IMU) data and optical-flow fusion. The stabilization models are configured to minimize motion artifacts during image acquisition and process. The camera 180 comprises a high-resolution coupled to the collimation assembly.

In one embodiment, a method of operation of the robotic surgical visualization system 100 is disclosed. At one step, the processing unit 108 is configured to guide the optic imaging probe through the robotic control unit 106 based on the user input provided via the user device 102. At another step, the processing unit 108 is further configured to actuate the robotic control unit 106 using a rack-and-pinion mechanism to enable pivoting of the imaging probe about the atraumatic unit 186. The actuation of the robotic control unit 106 is controlled manually via the user device 102, as well as hands-free through the wearable device 104. The sensors of the wearable device 104 transmit the control signals to the system based on the head movement and eye movements of the user. Further, the signals from the sensor enable intuitive probe control and adaptive image presentation.

At another step, the processing unit 108 is further configured to capture the stereoscopic image using the optic imaging probe positioned within the surgical area. At yet another step, the processing unit 108 is further configured to transmit the stereoscopic images to the camera 180 via the stereoscopic system. At yet another step, the processing unit 108 is configured to receive the stereoscopic images from the camera 180. At yet another step, the wearable device 104 is configured to enhance the captured images in real time using the artificial intelligence (AI) module. The artificial intelligence (AI) module is configured to enhance contrast, reduce visual noise, stabilize motion, or highlight anatomical features in real-time. At yet another step, the wearable device 104 is further configured to process the stereoscopic image via the visual-enhancement unit to enhance visual clarity and depth perception. At yet another step, the wearable device 104 is further configured enable the display of the processed images to the user via the display unit.

Advantageously, the system 100 is configured to provide high-resolution, real-time stereoscopic imagery during the microsurgical procedures. The system 100 integrates the robotic control unit 106, the fiber optic imaging probe, the artificial intelligence, and immersive display technologies to enhance the precision, clarity, and control of surgical visualization. The robotic control system 106 is configured to enable precise and stable manipulation of the microfiber probe within the surgical area. The robotic control system 106 is accomplished through advanced motion control algorithms that facilitate high-precision, tremor-free probe movement, enhancing surgical accuracy and safety. The stereoscopic system is designed to provide imaging capabilities compatible with the small diameters of modern microsurgical instrumentation, including 20 gauge (G), 23G, 25G, and 27G vitrectomy systems.

In one embodiment, the system 100 is designed as a modular unit that could be integrated with commercially available light collimation systems. The wearable device 104 is the handheld controller. The handheld controller is a manual controller that allows the surgeon to directly guide the movement of the microfiber probe within the surgical cavity. The surgeon might wear the headset for visualization while using the handheld controller for manual input and enabling tailored control. The system 100 is designed to deliver high-resolution imaging from the tip of the fiber optic bundle 142 with minimal diameter, enabling deep anatomical access while preserving image quality. The components within the lens adjustment assembly 182 includes the diffuser arrays and beam expansion optics serve to evenly illuminate the surgical field and reduce optical artifacts. The resulting image transmitted via the dual channels of the stereoscopic system provides a highly detailed and depth-rich view essential for microsurgical precision.

One aspect of the present disclosure is directed to a robotic surgical visualization system comprising: (a) at least one user device associated with a user, wherein the user device is configured to provide a user input, wherein the user input provides information related to directional movement control, rotational orientation control, control input, and tactile feedback control; (b) at least one wearable device; (c) a modular robotic control unit, comprising (i) a base platform comprises one or more first apertures, wherein the first aperture is configured to receive an external surgical system; (ii) at least one first base disposed on the base platform comprise a first sliding member and a second sliding member, wherein the first sliding member and the second sliding member comprises at least one groove, (iii) a first housing comprising: (iiia) at least one second base positioned on the first base comprise at least one first cavity and one or more appendages, wherein the first cavity comprises a first rack gear and a recessed channel configured to follow the curvature of the path and allow atraumatic movement of the robotic control unit about a center point of the atraumatic unit, wherein the one or more appendages are configured on a bottom surface of the second base, wherein the one or more appendages are configured to secure within the first groove of the first sliding member and the second sliding member; (iiib) a supporting member disposed on the second base comprises one or more second cavities at a rear portion of the supporting member; wherein at least one second cavity is positioned at a lower portion of the supporting member, and at least one second cavity is positioned at an upper portion of the supporting member, (iiic) one or more first motors positioned within the second cavities of the supporting member, wherein the supporting member comprises one or more second apertures for one or more wires and connections required for the system along with a honeycomb structure for material savings, and strength, and (iiid) a first pinion gear is configured to position along the first sliding member, wherein the first pinion gear is engaged with the first motor configured to transmit rotational motion, wherein the first pinion gear positioned with the first rack gear is configured to convert rotational motion from the motor into linear motion. In one embodiment, the system further comprises (a) a second housing is disposed on the first housing comprising: (i) a frame member comprises one or more track channels, an anterior end and a posterior end opposite to the anterior end, wherein the anterior end is connected to a probe guide and the posterior end is configured to receive a guide system, wherein the guide system comprises a probe tunnel, a circular track, one or more third apertures, and one or more pitch control motors, (ii) one or more fiber optic bundles run along the supporting member extend from the posterior end of the frame member and pass through the anterior end of the frame member, wherein the fiber optic bundle is configured to transmit an optical signal and an image data, (iii) a stereoscopic system connected to the frame member comprises one or more optic imaging probe, a collimation assembly, and one or more cameras, wherein the optic imaging probe coupled to the fiber optic bundle configured to capture a real-time stereoscopic image of a surgical area, wherein the camera is configured to convert transmitted optical signals into digital imagery and processing the stereoscopic images using a computing unit to enhance visual clarity and depth perception; and (b) a processing unit disposed on the base platform is in communication with the user device and the wearable device, wherein the processing unit is configured to receive control signals and the user input from the user device and the wearable device, wherein the processing unit is configured to provide output control signals to motors, wherein the processing unit is configured to receive the stereoscopic image from the robotic control unit and transmit real-time stereoscopic image to the wearable device, wherein the processing unit is configured to store calibration data, manage communication with the user device and the wearable device, and coordinate feedback loops for closed-loop control of the system.

The aperture of the base may be configured to attach to existing surgical robotic systems/arms. For example, in one embodiment, the system may be configured to attach to the distal appendages of the DEXTER® Robotic Surgery System (Distalmotion Inc, Orange Village, OH). In another embodiment, the system may be configured to interface with the distal appendages of the Hugo™ Robotic-Assisted Surgery System (Medtronic, Minneapolis, MN).

If a user or client desires to use our system with an existing surgical robot, the aperture of the system's base is designed to interface with these systems to allow for additional degrees of freedom while allowing the system to provide precise visualization capabilities. In one embodiment, the system may be integrated with the Hugo™ Robotic-Assisted Surgery System (Medtronic, Minneapolis, MN). In such an embodiment, grooves or channels may be incorporated into the system's base corresponding to the exact receiving dimensions of the of the third-party system's distal appendages, which under normal operation typically accommodate interchangeable instrumentation cartridges. Once physically integrated into the third-party machine, no additional digital connections to the external system are required, and the user may operate both systems independently.

In one embodiment, a user may desire to use only the core visualization components of the present system with the motor control and manipulation capabilities of an existing surgical system for certain applications in which atraumatic manipulation of the probe is of less importance. In such embodiments, the user may use the entire upper frame, including the housing, supports, and all internal visualization components (probe, fibers, lens collimation system, cameras, wires, and processing unit) to interface with the arm of an existing surgical system. In one embodiment, the system may be integrated with the Hugo™ Robotic-Assisted Surgery System (Medtronic, Minneapolis, MN). In such an embodiment, grooves or channels may be incorporated into the inferior aspect of the system's upper frame corresponding to the exact receiving dimensions of the of the third-party system's distal appendages, which under normal operation typically accommodate interchangeable instrumentation cartridges. Once physically integrated into the third-party machine, no additional digital connections to the external system are required, and the user may operate both systems independently. In such embodiments, the sole change from a user experience perspective is that axial motor control will be handled by the external system, though vertical control of the probe is retained by the presently disclosed system (through the pitch control motors contained in the upper frame). In such embodiments, the described imaging processing workflow, from distal fibers to processing unit and wearable headset, is completely retained.

In one embodiment, the probe tunnel comprises a second rack gear, wherein the second rack gear interfaces with the second pinion gear to control of the movement of the guide system, wherein the probe tunnel is configured to end at the probe guide, wherein the probe tunnel is configured to receive an auxiliary system to support the system, wherein the auxiliary system comprises lighting, suction, or irrigation. In another embodiment, each third aperture is configured to receive a threaded component, wherein the threaded component is configured to secure the guide system to the frame member via one or more nuts, thereby enabling the frame member to move along one dimension with minimal friction.

In one embodiment, the system further comprises a first connector configured to the appendages and the groove, wherein the first connector is configured to provide curvilinear movement between the appendages and the groove, wherein the first connector is configured to reduce friction and provides stability during motor operation and repositioning of the motor. In another embodiment, the appendage comprises a damper and a spring-loaded mechanism, wherein the appendages is configured to absorb shocks and vibrations induced during the movement.

In one embodiment, the wearable device comprises a display unit, one or more sensors, and a visual-enhancement unit, wherein the display unit is configured to display the captured stereoscopic images in real time, wherein the display unit is configured to enable users to remotely control the robotic control unit to navigate the optic imaging probe via the wearable device. In another embodiment, the sensor comprises a motion tracking sensor configured to detect a head movement and an eye movement of the user. In a related embodiment, the wearable device is configured to provide hands free control functionality by translating the detected head movement into a control signal, wherein the control signals are configured to adjust the position and orientation of the optic imaging probe, wherein the hands-free control functionality enables intuitive, real-time navigation within the surgical environment.

In one embodiment, the user device is a handheld controller, wherein the user device is configured to control the movement and motion of the robotic control unit. In another embodiment, the circular track is configured to receive a second pinion gear, wherein the second pinion gear is engaged with at least one first motor configured to transmit rotational motion. In one embodiment, the pitch control motor comprises a first pitch control motor and a second pitch control motor positioned adjacent to a flared opening at the posterior end of the frame member, wherein the positioning of the pitch control motors are configured to connect to one or more pitch control wires running along the visualization system, wherein the pitch control wire is configured to transmit the motion from the pitch control motors.

In one embodiment, the second housing comprises a gear track, wherein the gear track is configured to connect the pitch control motor and the first housing, thereby enabling pitch adjustment of the system, wherein the gear track is configured to provide guidance to prevent misalignment of the pitch control motor, and ensures consistent, reliable performance over an extended period of the pitch motor control, wherein the pitch control motor along the gear track enables bi-directional manipulation of the pitch control wires with high responsiveness. In another embodiment, the real-time stabilization module further comprises an inertial measurement unit (IMU) data and optical-flow fusion; wherein the stabilization models are configured to minimize motion artifacts during image acquisition and process.

In one embodiment, the probe guide comprises a probe body and a probe cover, wherein the probe body comprises an opening configured to allow the fiber optic bundle to pass through, and secure both the fiber optic bundle and the pitch control wire, wherein the probe cover is configured to cover both the fiber optic bundle and the pitch control wire, wherein the probe cover is configured to support and direct the optical pathways created by the fiber optic bundles. In another embodiment, the fiber optic bundles are configured to optimize light transmission, minimize optical aberrations and deliver high-resolution imaging at sub-millimeter scales, wherein the fiber optic bundles connected to the probe guide anteriorly and the fiber optic bundles connect posteriorly to the stereoscopic system. In one embodiment, the collimation assembly coupled to the fiber optic bundles, and the camera is configured to align the transmitted optical signals, wherein the collimation assembly comprises one or more collimating lenses, one or more aspheric lenses, one or more beam diffusers, one or more lens adjustable assembly, and one or more beam expansion optics, wherein the lens adjustment assemblies connected to the respective fiber optic bundles are configured to guide light from the surgical area of interest to the camera.

In one embodiment, the robotic control unit further comprises a disc member is configured to surround the fiber optic bundles and interfaces with the pitch control wires, wherein the disc member receives the force from the pitch control wire to bend the fiber optic bundles in the intended pitch direction, wherein the disc member is configured to provides a stable and aligned base for the routing of the fiber optic bundles and the pitch control wires. In another embodiment, the display unit comprises a communication interface with the visual-enhancement unit to dynamically adjust the display based on user movement.

In another embodiment of the system, the visual-enhancement unit comprises an artificial intelligence (AI) module and a real-time stabilization module, wherein the AI module is configured to: (a) analyze and optimize images; (b) generate a depth map from acquired stereoscopic image data in real time; (c) filter noise from the image data; (d) perform image sharpening, contrast enhancement, and edge detection in real time, and (e) overlay anatomical highlights onto the processed image in real time.

Another aspect of the present disclosure is directed to a method for robotic surgical visualization system for surgery comprising: (a) guiding, at the processing unit, the optic imaging probe through the robotic control unit based on the user input provided via the user device; (b) actuating, at the processing unit, the robotic control unit using a rack-and-pinion mechanism to enable pivoting of the imaging probe about an atraumatic unit, wherein the actuation of the robotic control unit is controlled manually via a user device, as well as hands-free through the wearable device, wherein the sensors of the wearable device transmits the control signals to the system based on the head movement and eye movements of the user, wherein the signals from the sensor enable for intuitive probe control and adaptive image presentation; (c) capturing, at the processing unit, the stereoscopic image using the optic imaging probe positioned within a surgical site; (d) transmitting, at the processing unit, the stereoscopic images to the camera via the stereoscopic system; (e) receiving, at the processing unit, the stereoscopic images from the camera; (f) enhancing, at the wearable device, the captured images in real time using the artificial intelligence (AI) module to enhance contrast, reduce visual noise, stabilize motion, and highlight anatomical features in real-time; (g) processing, at the wearable device, the stereoscopic image via the visual-enhancement unit to enhance visual clarity and depth perception, and (h) enabling, at the wearable device, display the processed images to a user via the display unit.

What is claimed is:

1. A robotic surgical visualization system comprising:
(a) at least one user device associated with a user, wherein the user device is configured to provide a user input, wherein the user input provides information related to directional movement control, rotational orientation control, control input, and tactile feedback control;
(b) at least one wearable device;
(c) a modular robotic control unit, comprising:
a. a base platform that includes one or more first apertures, wherein the first aperture is configured to receive an external surgical system,
b. at least one first base disposed on the base platform that includes a first sliding member and a second sliding member, wherein the first sliding member and the second sliding member comprises at least one groove; and
(d) a first housing, comprising:
a. at least one second base positioned on the first base comprising at least one first cavity and one or more appendages, wherein the first cavity comprises a first rack gear and a recessed channel configured to follow the curvature of the path and allow atraumatic movement of the robotic control unit about a center point of the atraumatic unit, wherein the one or more appendages are configured on a bottom surface of the second base, wherein the one or more appendages are configured to secure within the first groove of the first sliding member and the second sliding member;
b. a supporting member disposed on the second base comprising one or more second cavities at a rear portion of the supporting member; wherein at least one second cavity is positioned at a lower portion of the supporting member, and at least one second cavity is positioned at an upper portion of the supporting member;
c. one or more first motors positioned within the second cavities of the supporting member, wherein the supporting member comprises one or more second apertures for one or more wires and connections required for the system along with a honeycomb structure for material savings, and strength; and
d. a first pinion gear is configured to position along the first sliding member, wherein the first pinion gear is engaged with the first motor configured to transmit rotational motion, wherein the first pinion gear positioned with the first rack gear is configured to convert rotational motion from the motor into linear motion.

2. The system of claim 1, further comprising:
a. a second housing disposed on the first housing, comprising:
i. a frame member comprises one or more track channels, an anterior end and a posterior end opposite to the anterior end, wherein the anterior end is connected to a probe guide and the posterior end is configured to receive a guide system, wherein the guide system comprises a probe tunnel, one or more third apertures, and one or more pitch control motors,
ii. one or more fiber optic bundles run along the supporting member extend from the posterior end of the frame member and pass through the anterior end of the frame member, wherein the fiber optic bundle is configured to transmit an optical signal and an image data, and
iii. a stereoscopic system connected to the frame member comprising one or more optic imaging probes, a collimation assembly, and one or more cameras, wherein the optic imaging probe coupled to the fiber optic bundle is configured to capture real-time stereoscopic images of a surgical area, wherein the camera is configured to convert transmitted optical signals into digital imagery and processing the stereoscopic images using a computing unit to enhance visual clarity and depth perception; and
b. a processing unit disposed on the base platform is in communication with the user device and the wearable device, wherein the processing unit is configured to receive control signals and the user input from the user device and the wearable device, wherein the processing unit is configured to provide output control signals to motors, wherein the processing unit is configured to receive the stereoscopic image from the robotic control unit and transmit real-time stereoscopic image to the wearable device, wherein the processing unit is configured to store calibration data, manage communication with the user device and the wearable device, and coordinate feedback loops for closed-loop control of the system.

3. The system of claim 1, wherein the probe tunnel comprises a second rack gear, wherein the second rack gear interfaces with the second pinion gear to control of the movement of the guide system, wherein the probe tunnel is configured to end at the probe guide, wherein the probe tunnel is configured to receive an auxiliary system to support the system, wherein the auxiliary system comprises lighting, suction, or irrigation.

4. The system of claim 1, wherein each third aperture is configured to receive a threaded component, wherein the threaded component is configured to secure the guide system to the frame member via one or more nuts, thereby enabling the frame member to move along one dimension with minimal friction.

5. The system of claim 1, further comprises a first connector configured to the appendages and the groove, wherein the first connector is configured to provide curvilinear movement between the appendages and the groove, wherein the first connector is configured to reduce friction and provides stability during motor operation and repositioning of the motor.

6. The system of claim 1, wherein the appendage comprises a damper and a spring-loaded mechanism, wherein the appendages is configured to absorb shocks and vibrations induced during the movement.

7. The system of claim 1, wherein the wearable device comprises a display unit, one or more sensors, and a visual-enhancement unit, wherein the display unit is configured to display the captured stereoscopic images in real time, wherein the display unit is configured to enable users to remotely control the robotic control unit to navigate the optic imaging probe via the wearable device.

8. The system of claim 4, wherein the sensor comprises a motion tracking sensor configured to detect a head movement and an eye movement of the user.

9. The system of claim 4, wherein the wearable device is configured to provide hands free control functionality by translating the detected head movement into a control signal, wherein the control signals are configured to adjust the position and orientation of the optic imaging probe, wherein the hands-free control functionality enables intuitive, real-time navigation within the surgical environment.

10. The system of claim 1, wherein the user device is a handheld controller, wherein the user device is configured to control the movement and motion of the robotic control unit.

11. The system of claim 1, wherein the circular track is configured to receive a second pinion gear, wherein the second pinion gear is engaged with at least one first motor configured to transmit rotational motion.

12. The system of claim 1, wherein the pitch control motor comprises a first pitch control motor and a second pitch control motor positioned adjacent to a flared opening at the posterior end of the frame member, wherein the positioning of the pitch control motors are configured to connect to one or more pitch control wires running along the visualization system, wherein the pitch control wire is configured to transmit the motion from the pitch control motors.

13. The system of claim 1, wherein the second housing comprises a gear track, wherein the gear track is configured to connect the pitch control motor and the first housing, thereby enabling pitch adjustment of the system, wherein the gear track is configured to provide guidance to prevent misalignment of the pitch control motor, and ensures consistent, reliable performance over an extended period of the pitch motor control, wherein the pitch control motor along the gear track enables bi-directional manipulation of the pitch control wires with high responsiveness.

14. The system of claim 1, wherein the real-time stabilization module further comprises an inertial measurement unit (IMU) data and optical-flow fusion; wherein the stabilization models are configured to minimize motion artifacts during image acquisition and process,

[symbol]

15. The system of claim 1, wherein the probe guide comprises a probe body and a probe cover, wherein the probe body comprises an opening configured to allow the fiber optic bundle to pass through, and secure both the fiber optic bundle and the pitch control wire, wherein the probe cover is configured to cover both the fiber optic bundle and the pitch control wire, wherein the probe cover is configured to support and direct the optical pathways created by the fiber optic bundles.

16. The system of claim 1, wherein the fiber optic bundles are configured to optimize light transmission, minimize optical aberrations and deliver high-resolution imaging at sub-millimeter scales, wherein the fiber optic bundles connected to the probe guide anteriorly and the fiber optic bundles connect posteriorly to the stereoscopic system.

17. The system of claim 1, wherein the collimation assembly coupled to the fiber optic bundles, and the camera is configured to align the transmitted optical signals, wherein the collimation assembly comprises one or more collimating lenses, one or more aspheric lenses, one or more beam diffusers, one or more lens adjustable assembly, and one or more beam expansion optics, wherein the lens adjustment assemblies connected to the respective fiber optic bundles are configured to guide light from the surgical area of interest to the camera.

18. The system of claim 1, wherein the robotic control unit further comprises a disc member is configured to surround the fiber optic bundles and interfaces with the pitch control wires, wherein the disc member receives the force from the pitch control wire to bend the fiber optic bundles in the intended pitch direction, wherein the disc member is configured to provides a stable and aligned base for the routing of the fiber optic bundles and the pitch control wires.

19. The system of claim 1, wherein the display unit comprises a communication interface with the visual-enhancement unit to dynamically adjust the display based on user movement.

20. The system of claim 1, wherein the visual-enhancement unit comprises an artificial intelligence (AI) module and a real-time stabilization module, wherein the AI module is configured to:
(a) analyze and optimize images;
(b) generate a depth map from acquired stereoscopic image data in real time;
(c) filter noise from the image data;
(d) perform image sharpening, contrast enhancement, and edge detection in real time; and
(e) overlay anatomical highlights onto the processed image in real time.

21. A method for robotic surgical visualization system for surgery comprising:
(a) guiding, at the processing unit, the optic imaging probe through the robotic control unit based on the user input provided via the user device;
(b) actuating, at the processing unit, the robotic control unit using a rack-and-pinion mechanism to enable pivoting of the imaging probe about an atraumatic unit, wherein the actuation of the robotic control unit is controlled manually via a user device, as well as hands-free through the wearable device, wherein the sensors of the wearable device transmits the control signals to the system based on the head movement and eye movements of the user, wherein the signals from the sensor enable for intuitive probe control and adaptive image presentation;
(c) capturing, at the processing unit, the stereoscopic image using the optic imaging probe positioned within a surgical site;
(d) transmitting, at the processing unit, the stereoscopic images to the camera via the stereoscopic system;
(e) receiving, at the processing unit, the stereoscopic images from the camera;
(f) enhancing, at the wearable device, the captured images in real time using the artificial intelligence (AI) module to enhance contrast, reduce visual noise, stabilize motion, and highlight anatomical features in real-time;
(g) processing, at the wearable device, the stereoscopic image via the visual-enhancement unit to enhance visual clarity and depth perception; and
(h) enabling, at the wearable device, display the processed images to a user via the display unit.
